(19) United States
(12) Patent Application Publication
(10) Pub. No.: US 2002/0065263 A1
(43) Pub. Date: May 30, 2002

(54) Formulation

(75) Inventors: Africa Aranda Garcia, Toledo (ES); Fernando Hijar Ordovas, Madrid (ES)

Correspondence Address: GLAXOSMITHKLINE, Corporate Intellectual Property - UW2220, P.O. Box 1539, King of Prussia, PA, 19406-0939, US

(73) Assignee: SmithKline Beecham S.A.

(21) Appl. No.: 09/998,404

(22) Filed: Nov. 30, 2001

Related U.S. Application Data
(63) Continuation of application No. 09/548,810, filed on Apr. 14, 2000.

(30) Foreign Application Priority Data
Apr. 16, 1999 (GB) GB 9908776.9

(51) Int. Cl. A61K 31/545

(52) U.S. Cl. 514/204

(57) ABSTRACT

A new parenteral dosage form for 7-D-mandelamido-3-(1-sulfomethyltetrazol-5-yl)thiomethyl) -3-cephem-4-carboxylic acid mono sodium salt (“Cefonicid”) is provided.

This invention relates to a novel pharmaceutical formulation comprising 7-D-mandelamido-3-(1-sulfomethyltetrazol-5-yl)thiomethyl)-3-cephem-4-carboxylic acid, a method for the preparation of such a formulation and its use as an anti-bacterial agent.

U.S. Pat. No. 4,083,311 discloses the compound 7-D-mandelamido-3-(1-sulfomethyltetrazol-5-yl)thiomethyl) -3-cephem-4-carboxylic acid (“Cefonicid”), that is to say the compound of formula (I),

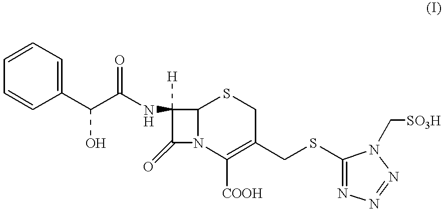

as being a novel cephalosporin compound which possesses anti-bacterial activity. Example 1 relates to the preparation of this compound, which is isolated and analysed as its di-sodium salt. European Patent application 0154484A2 discloses that the monosodium salt of this compound exhibits a higher level of thermal stability than the di-sodium salt, whilst still retaining its anti-bacterial properties. Example 2 relates to an injectable pharmaceutical composition formed by adding sterile water or sterile saline solution to the monosodium salt of this compound.

A new parenteral formulation has now been discovered, having improved properties as compared with conventional formulations. The present invention therefore provides, in a first aspect, a parenteral pharmaceutical composition which comprises 7-D-mandelamido-3-(-sulfomethyltetrazol-5-yl)thiomethyl)-3-cephem-4-carboxylic acid monosodium salt in a buffered aqueous solution.

Advantageously, the solution is to be buffered at a pH range of 4.5-5.5. It will be appreciated by persons skilled in the art that the choice of buffer reagents can materially affect the efficacy of the active compound. Typical buffering reagents for use in the formulations of this invention include sodium acetate-acetic acid di-sodium phosphate-ti-sodium phosphate, sodium carbonate and tri-sodium citrate-citric acid. Most preferably the buffered aqueous solution comprises 12.0-12.3% w/v tri-sodium citrate and 0.35-0.40% w/v citric acid monohydrate.

The pharmaceutically active compound 7-D-mandelamido-3-(1-sulfomethyltetrazol-5-yl)thiomethyl) -3-cephem-4-carboxylic acid monosodium salt will be present in an amount from 500-2000 mgs. per dosage form.

The administration of the formulation of this invention is advantageously made by parenteral injection such as subcutaneously, intramuscularly or intravenously. For intramuscular injection the formulation will typically also include a local anaesthetic such as lidocaine hydrochloride (Acetamide, 2-(diethylamino)-N-(2,6-dimethylphenyl)-hydrochloride).

The present invention further provides a method for the preparation of a pharmaceutical formulation, as hereinbefore defined, which comprises adding an aqueous solution comprising buffering agents, suitable for maintaining the formulation in the pH range of 4.5-5.5, to 7-D-mandelamido-3-(1-sulfomethyltetrazol-5-yl)thiomethyl)-3-cephem-4-carboxylic acid monosodium salt and mixing thoroughly until the active agent has fully dissolved.

In a yet further aspect, the invention provides a kit comprising a dosage unit of 7-D-mandelamido-3-(1-sulfomethyltetrazol-5-yl)thiomethyl)-3-cephem-4-carboxylic acid monosodium salt and an aqueous solution comprising buffering agents, suitable for maintaining the composition in the pH range of 4.5-5.5. Examples of suitable buffering agents are as described above.

The present invention further provides a method of treatment of bacterial infections which comprises administering to a host in need thereof an effective but non-toxic amount of a pharmaceutical composition as described above.

The following examples are not limiting but are merely illustrative of the formulations of this invention.

### EXAMPLE 1

### Intravenous Pharmaceutical Composition

A preparation which contains 1000 mg of 7-D-mandelamido-3-(1-sulfomethyltetrazol-5-yl)thiomethyl) -3-cephem-4-carboxylic acid monosodium salt for parenteral injection was prepared in the following manner. An ampoule comprising 548.4 mg of tri-sodium citrate and 16.50 mg of citric acid monohydrate in 4.5 ml of water was prepared. The resulting solution was then added to vial containing 1000 mg of 7-D-mandelamido-3-(1-sulfomethyltetrazol-5-yl)thiomethyl) -3-cephem-4-carboxylic acid monosodium salt and the resulting solution stirred until the active compound had fully dissolved. The resulting solution was determined to have a pH of 4.6.

The following formulations were prepared in a similar manner to that of Example 1. Lidocaine hydrochloride was also added for intra-muscular compositions.

| Example 2 | | |
|---|---|---|
| Intramuscular Pharmaceutical Formulation | | |
| Vial: 1000 mg monosodium salt | | |
| Ampule: 4.5 ml | | |
| lidocaine hydrochloride: | 30.00 | mg |
| tri-sodium citrate: | 548.4 | mg |
| citric acid monohydrate | 16.5 | mg |
| Example 3 | | |
| Intravenous Pharmaceutical Formulation | | |
| Vial: 1000 mg monosodium salt | | |
| Ampule: 3 ml or 4.5 ml | | |
| sodium acetate dihydrate: | 369.00 | mg |
| acetic acid: | 17.40 | mg |
| Example 4 | | |
| Intramuscular Pharmaceutical Formulation | | |
| Vial: 1000 mg monosodium salt | | |
| Ampule: 3 ml or 4.5 ml | | |
| sodium acetate dihydrate: | 369.00 | mg |
| acetic acid: | 17.40 | mg |
| lidocaine hydrochloride: | 30.00 | mg |
| Example 5 | | |
| Intravenous Pharmaceutical Formulation | | |
| Vial: 1000 mg monosodium salt | | |
| Ampule: 3 ml or 4.5 ml | | |
| di-sodium phosphate: | 30.0 | mg |
| tri-sodium phosphate: | 276.0 | mg |
| Example 6 | | |
| Intravenous Pharmaceutical Formulation | | |
| Vial: 1000 mg monosodium salt | | |
| Ampule: 3 ml or 4.5 ml | | |
| disodium phosphate: | 30.0 | mg |
| trisodium phosphate: | 360.0 | mg |
| Example 7 | | |
| Intravenous Pharmaceutical Formulation | | |
| Vial: 1000 mg monosodium salt | | |
| Ampule: 3 ml or 4.5 ml | | |
| disodium phosphate: | 30.0 | mg |
| trisodium phosphate: | 300.0 | mg |
| Example 8 | | |
| Intravenous Pharmaceutical Formulation | | |
| Vial: 1000 mg monosodium salt | | |
| Ampule: 3 ml or 4.5 ml | | |
| Sodium carbonate: | 120.0 | mg |
| Example 9 | | |
| Intravenous Pharmaceutical Formulation | | |
| Vial: 1000 mg monosodium salt | | |
| Ampule: 3 ml or 4.5 ml | | |
| Sodium carbonate: | 135.0 | mg |
| Example 10 | | |
| Intravenous Pharmaceutical Formulation | | |
| Vial: 1000 mg monosodium salt | | |
| Ampule: 3 ml or 4.5 ml | | |
| Sodium carbonate: | 142.5 | mg |
| Example 11 | | |
| Intramuscular Pharmaceutical Formulation | | |
| Vial: 1000 mg monosodium salt | | |
| Ampule: 3 ml or 4.5 ml | | |
| sodium acetate dihydrate: | 624.0 | mg |
| acetic acid monohydrate: | 15.0 | mg |
| lidocaine hydrochloride: | 30.00 | mg |
| Example 12 | | |
| Intramuscular Pharmaceutical Formulation | | |
| Vial: 1000 mg monosodium salt | | |
| Ampule: 3 ml or 4.5 ml | | |
| sodium acetate dihydrate: | 549.9 | mg |
| acetic acid monohydrate: | 15.0 | mg |
| lidocaine hydrochloride: | 30.00 | mg |

1. A parenteral pharmaceutical formulation which comprises 7-D-mandelamido-3-(1-sulfomethyltetrazol-5-yl)thiomethyl)-3-cephem-4-carboxylic acid monosodium salt in a buffered aqueous solution.

2. A pharmaceutical formulation according to claim 1 in which the solution is buffered at a pH range of 4.5-5.5.

3. A pharmaceutical formulation according to claim 1 or 2 in which the buffered aqueous solution comprises buffering reagents selected from the group consisting of sodium acetate-acetic acid, disodium phosphate-trisodium phosphate, sodium carbonate and trisodium citrate-citric acid.

4. A pharmaceutical formulation according to claim 1 or 2 in which the buffered aqueous solution comprises 12.0-12.3% w/v trisodium citrate and 0.35-0.40 w/v citric acid monohydrate.

5. A method for the preparation of a pharmaceutical formulation according to claim 1 which comprises adding an aqueous solution comprising buffering agents suitable for maintaining the formulation in the pH range of 4.5-4.9 to 7-D-mandelamido-3-(1-sulfomethyltetrazol-5-yl)thiomethyl) -3-cephem-4-carboxylic acid monosodium salt and mixing thoroughly until the active agent has fully dissolved.

6. A kit comprising a dosage unit of 7-D-mandelamido-3-(1-sulfomethyltetrazol-5-yl)thiomethyl) -3-cephem-4-carboxylic acid monosodium salt and an aqueous solution comprising buffering agents suitable for maintaining the formulation in the pH range of4.5-5.5.

7. A method of treatment of bacterial infections which comprises administering to a host in need thereof an effective but non-toxic amount of a parenteral formulation as claimed in claim 1.
